(19) United States
(12) Patent Application Publication
(10) Pub. No.: US 2020/0122628 A1
(43) Pub. Date: Apr. 23, 2020

(54) MULTI-FUNCTIONAL MOVIE INDUSTRY TRAILER SYSTEM AND METHOD

(71) Applicants: John Ekonomou, Port Coquitlam (CA); Chris Ekonomou, Port Coquitlam (CA)

(72) Inventors: John Ekonomou, Port Coquitlam (CA); Chris Ekonomou, Port Coquitlam (CA)

(21) Appl. No.: 16/175,588

(22) Filed: Oct. 30, 2018

(30) Foreign Application Priority Data
Oct. 18, 2018 (CA) 3,021,157

(51) Int. Cl. B60P 3/32; B62D 63/08; A61F 17/00

(52) U.S. Cl. CPC B60P 3/32; A61F 17/00; B62D 63/08

(57) ABSTRACT

A multi-functional movie industry trailer system including: a frame, an enclosure shell having at least one interior room, electrical wiring, and plumbing infrastructure; wherein the at least one interior room has at least two washrooms and a first-aid-room. The multi-functional movie industry trailer system includes in functional combination the frame upon which the enclosure shell is mounted. The enclosure shell has the at least one interior room, the at least two washrooms and the first-aid-room serviced by the electrical wiring and the plumbing infrastructure such that movie-related personnel can remain on-site.

### BACKGROUND OF THE INVENTION

This application claims benefit under 35 USC 119 to Canada National Application No. 3,021,157 filed on Oct. 17, 2018, the disclosure of which is incorporated herein by reference in its entirety. The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of land vehicles: bodies and tops of existing art and more specifically relates to land vehicles: bodies and tops having tent ground engaging means.

### RELATED ART

Many movies get made each year. Movie location sets are typically very crowded and space is at a premium. Actors, directors and staff all need separate trailers making this space problem even worse. Most location sets now require a first aid station/trailer in addition to what is already being used for safety and insurance issues. Storage for water/sewage requires space as hookups to utilities such as water sewer and power are often limited. Many movie sets require trailers for actors, directors and other staff that may or may not have washroom facilities but do not have first aid facilities. A suitable solution is desired.

Foreign Pat. No. DE2423561 to Hans Koenig relates to a toilet and washroom for caravan with pull-out design to increase room and with external access to service wc. The described toilet and washroom for caravan with pull-out design includes toilet and washing facilities mounted in a small compartment in the caravan. This can be increased in size, when the van is parked, by pulling out an extending wall. The pull-out section is fitted with access flaps to reach the w.c. and the waste water tank, as well as the fresh water tank, as well as the fresh water tank, from the outside of the van. This makes the servicing of the toilet facility cleaner and simpler. For added compactness the w.c. is sited under the wash basin which is hinged to swing upwards to gain access to the w.c.

### SUMMARY OF THE INVENTION

In view of the foregoing disadvantages inherent in the known land vehicles: bodies and tops art, the present disclosure provides a novel multi-functional movie industry trailer system and method. The general purpose of the present disclosure, which will be described subsequently in greater detail, is to provide a multi-functional trailer for use on movie sets.

The present invention is a movie industry trailer that combines washrooms with at least first aid facilities. The combination washroom/first aid trailer system is beneficial and saves valuable space on set. The trailer combines multiple washroom and first aid facilities in one trailer and may combine living quarters, washroom and first aid into one unit rather than 3 separate trailer units. This saves valuable space on movie set locations where space is at a premium.

A multi-functional movie industry trailer system is disclosed herein comprising: a frame, an enclosure shell having at least one interior room, electrical wiring, and plumbing infrastructure; wherein the at least one interior room comprises at least two washrooms and a first-aid-room. The multi-functional movie industry trailer system comprises in functional combination the frame upon which the enclosure shell is mounted. The enclosure shell comprises the at least one interior room, the washroom and the first-aid-room serviced by the electrical wiring and the plumbing infrastructure such that movie-related personnel can remain on-site.

A method of use for the multi-functional movie industry trailer system is also disclosed herein, the method comprising the steps of: 1) providing a multi-functional movie industry trailer system comprising: a frame, an enclosure shell having at least one interior room, electrical wiring, and plumbing infrastructure; wherein the at least one interior room comprises at least two washrooms and a first-aid-room, and 2) using the multi-functional movie industry trailer system onsite for washroom-use and first-aid-use. In preferred embodiments the multi-functional movie industry trailer system is mobile.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a multi-functional movie industry trailer system, constructed and operative according to the teachings of the present disclosure.

FIG. 1 is a perspective block view of the multi-functional movie industry trailer system during an ‘in-use’ condition, according to an embodiment of the disclosure.

FIG. 2 is a perspective block view of the multi-functional movie industry trailer system, according to an alternate embodiment of the present disclosure.

FIG. 3 is a perspective block view of the multi-functional movie industry trailer system, according to an alternate embodiment of the present disclosure.

FIG. 4 is a plan view of the multi-functional movie industry trailer system of FIG. 1, according to an embodiment of the present disclosure.

FIGS. 4B, 4C, 4D, 4E and 4F are plan views of alternate layouts of the multi-functional movie industry trailer system of FIG. 1.

FIG. 5 is a flow diagram illustrating a method of use for the multi-functional movie industry trailer system, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to land vehicles: bodies and tops and more particularly to a multi-functional movie industry trailer system as used to improve the efficiency of trailer use for movie industry personnel.

Generally, the trailer combines washroom and first aid facilities in one trailer and may also combine living quarters, washroom and first aid into one unit rather than 3 separate trailer units. This saves valuable space on movie set locations where space is at a premium. It is more economical to have one unit rather than 2 or 3 for each function, thus the present invention serves to promote efficiency and increase production. The trailer in preferred embodiments has three exterior entrances (First aid, male w/c, female w/c) and may also have interior doors connecting the first aid area to either washroom or to its own washroom (which may include a shower stall and/or tub). Also, there may be an extendible portion, for the first aid area, which can be extended over the tongue of the trailer (˜4 feet) to increase interior space when parked.

The present invention may comprise the following options:

Restroom With Sink And Toilet

Galley Single Bowl Kitchen Sink And Base Cab

Vinyl Covered Cot With Storage

Aluminum Wheels Per Wheel

Spare Tire. Mtg Kit & Cover—White Rim

Ram Stab Jacks IPO Scissor

Roof Mount 13.5K BTU Air Conditioner

Heat Strip For AC

64 Gallon Waste Tank 32×2

40 Gallon Fresh Water Tank

Winter Package With Meat Pads

Water Heater Electric 10 Gallon

interior 4′ 110V Fluorescent Light

Fire Extinguisher

Hard Window Valance—Each

Night: Shades Per Window

4×4 Heavy Duty Tongue Jack

Oversized 42″ Entrance Door

Fantastic Fan With Cover in Bathroom

Joy Connector

Fiberglass Walls IPO Standard

Fiberglass Cabinets IPO Standard

Stainless Steel Countertops IPO Standard

Eyewash & Kitchen Faucet Combo

Exterior light For Ramp

Stainless Steel Flip Up Countertop

Puck lights Under Cabinet—On Separate Switch

Double Pancake Ceiling lights With Switch—12V

Jet Tile Linoleum Flooring

750 W Electric Heater

Make Hitch Pintle

Add Brackets For Stretcher & O2 Storage

Add Decal Both Driver And Passenger Side “First Aid” W/Cross

Mako Dump Camlock

Note: Mount Spare Tire to Rear of Trailer

Make Entrance door 36

Omit 4×4 Heavy Duty Tongue Jack

Omit Fantastic Fan With Covar In Bathroom

Make Entrance door 32″ IPO 36″

White Spare Tire And Mounting Kit

Winter Pack With Heat Pads Under 19′ & Insulation

6 Gallon Electric Water Heater With Drain

Electric Room Heater 750 W With Wall Thermostat

Battery Shut Off Switch In Mechanical Room

Fresh Water 100 Gallons With Pump

Additional 50 Gallons To Make 150

Stereo With inside Speakers

Paper Towel Dispensers

Make Hitch Pintle

Camlock Front Dump

Add Joy Connector

Note: Decals—M, F, Unisex

Mount Spare Tire To The Back

Luxury Legacy Décor

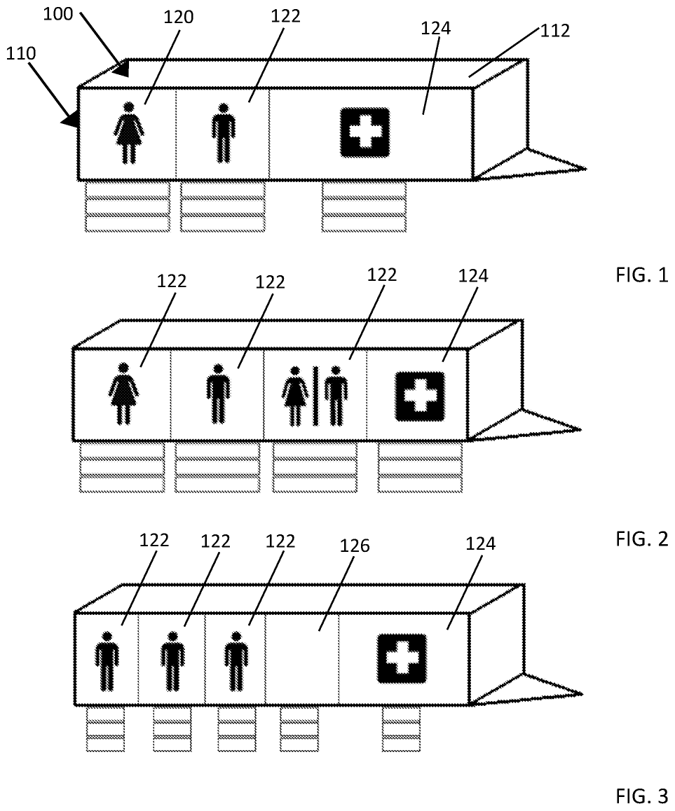

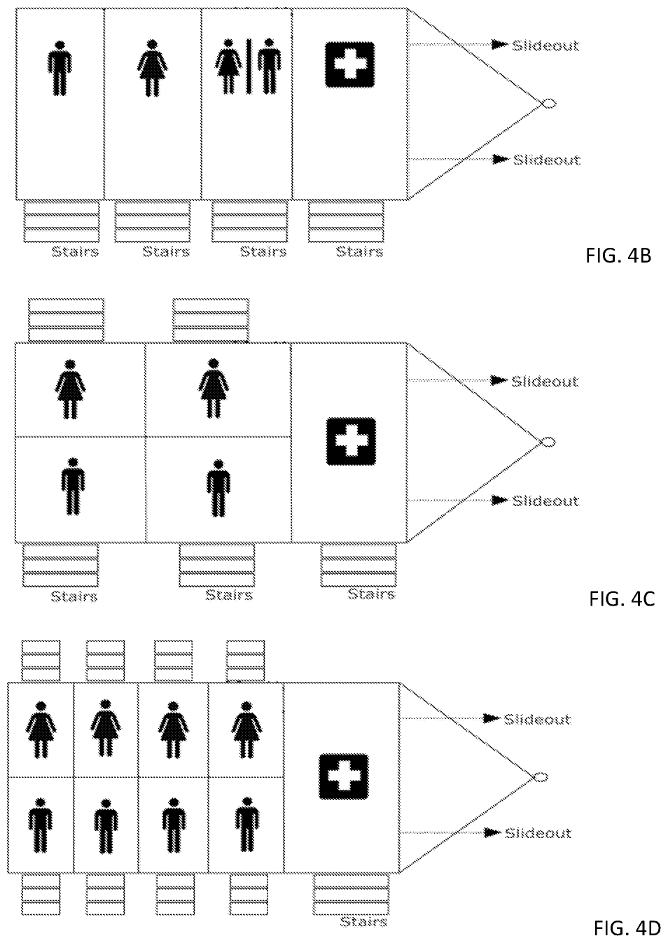

Referring now more specifically to the drawings by numerals of reference, there is shown in FIGS. 1-4, various views of a multi-functional movie industry trailer system 100.

FIG. 1 shows a multi-functional movie industry trailer system 100 during an ‘in-use’ condition 50, according to an embodiment of the present disclosure. Here, the multi-functional movie industry trailer system 100 may be beneficial for use by a user to create efficiency in trailer use on movie sets. As illustrated, the multi-functional movie industry trailer system 100 may include a frame 110, an enclosure shell 112 having at least one interior room 120, electrical wiring, and plumbing infrastructure; wherein the at least one interior room 120 comprises at least two washrooms 122 and a first-aid-room 124. The multi-functional movie industry trailer system 100 comprises in functional combination the frame 110 upon which the enclosure shell 112 is mounted. The enclosure shell 112 comprises the at least one interior room 120; the at least two washrooms 122 and the first-aid-room 124 serviced together by the electrical wiring and the plumbing infrastructure such that movie-related personnel can remain on-site and use the facilities while on-shift or after shift.

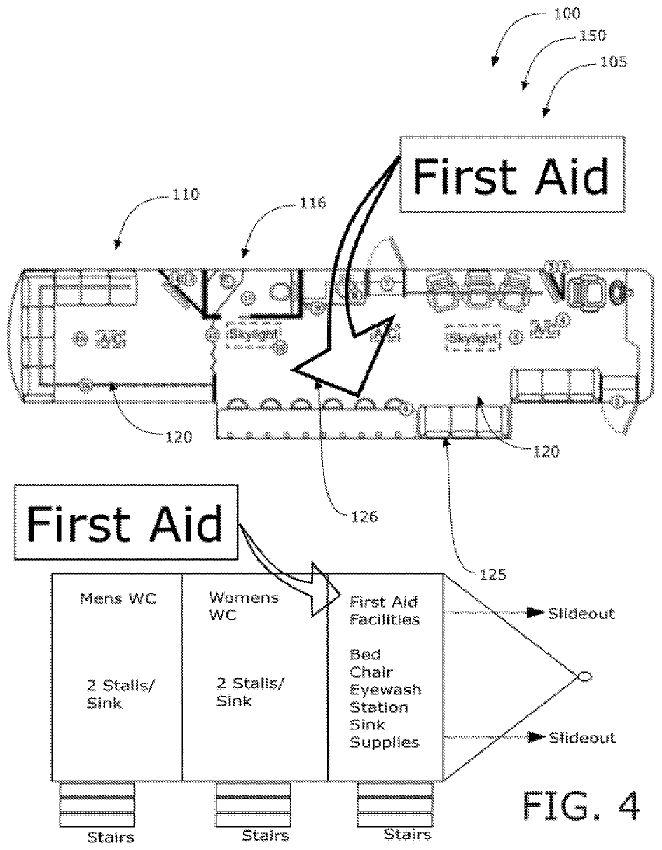

Referring now to FIGS. 2-4 showing various views of the multi-functional movie industry trailer system 100 of FIG. 1, according to an embodiment of the present disclosure.

The at least one interior room 120 of the multi-functional movie industry trailer system 100 may comprise at least the washrooms 122, the first-aid-room 124 and a living quarters 126 in certain embodiments. Washrooms 122 may have single stalls, multiple stalls, and one or more sinks, as required and depending on needs. Alternate embodiments may comprise other rooms such as storage rooms, waiting rooms, electrical rooms, and the like. The first-aid-room 124 preferably comprises first-aid-supplies 125. The first-aid-supplies 125 may comprise at least plasters in a variety of different sizes and shapes, small, medium and large sterile gauze dressings, at least 2 sterile eye dressings, triangular bandages, crepe rolled bandages, safety pins, disposable sterile gloves, tweezers, scissors, alcohol-free cleansing wipes, sticky tape, thermometer, skin rash cream, cream and alternately spray to relieve insect bites and stings, antiseptic cream, painkillers, cough medicine, antihistamine tablets, distilled water, and a basic first aid manual. Other safety and first-aid-supplies 125 may be included such as fire extinguishers, cots, slings, crutches, various medical devices, medicines and the like. The list has been provided as only an exemplary means and is no way considered to be exhaustive.

According to one embodiment, the multi-functional movie industry trailer system 100 may be arranged as a kit 105 for first-aid-supplies 125. In particular, the multi-functional movie industry trailer system 100 may further include a set of instructions. The instructions may detail functional relationships in relation to the structure of the multi-functional movie industry trailer system 100 such that the first-aid-supplies 125 of the multi-functional movie industry trailer system 100 can be used, maintained, or the like, in a preferred manner.

The enclosure shell 112 comprises exterior-walls 114, and interior-walls 116. The frame 110 may be stationary or the frame 110 may comprise axles and wheels mounted thereto (not shown). The frame 110 may comprise a hitch for moving the present invention between locations, as desired. The first-aid-room 124 is accessible via a door in the exterior-wall 114. The first-aid-room 124 may comprise bathes and wash stations; wherein the bathes and the wash stations are coupled to the plumbing infrastructure. The first aid room 124 may further comprise a shower stall coupled to the plumbing infrastructure. The bathes and wash stations may comprise valves, as needed. The plumbing infrastructure is provided with holding tanks for water and sewage purposes, which may be filled and emptied as required. The plumbing infrastructure may further include connections to be hooked temporarily to water and sewage connections where and when available and may be used to transfer at least water. The first-aid-room 124 preferably comprises shelving. The electrical wiring is connected into the first-aid-room 124. Those with ordinary skill in the art will now appreciate that upon reading this specification and by their understanding the art of trailers, hookups to utilities, first-aid equipment and storage as described herein, methods of making the present invention efficient will be understood by those knowledgeable in such art.

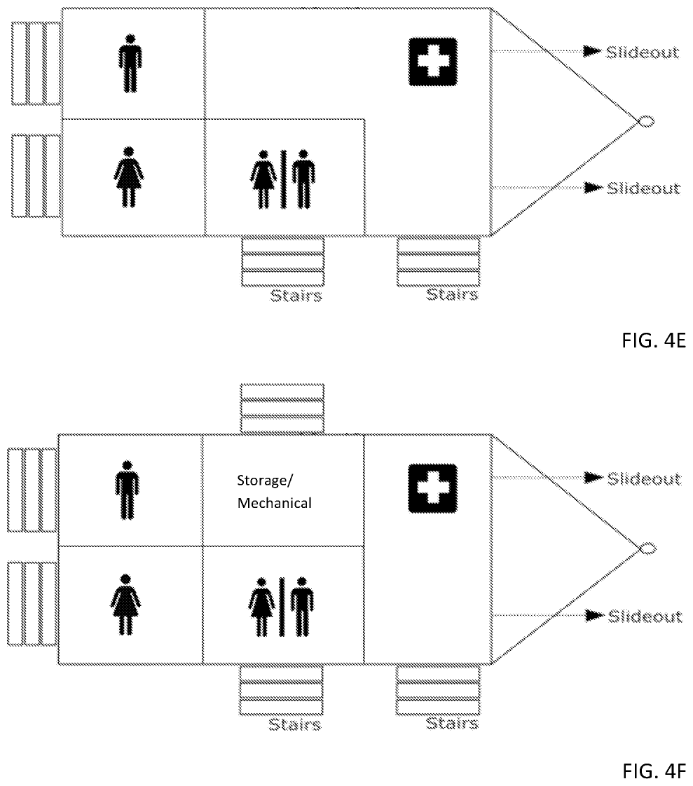

FIGS. 4B, 4C and 4D show alternative layouts with multiple stalls and access locations within the trailer system 100. More particularly, FIG. 4B shows a layout with three washroom stalls (male, female, unisex) and a first aid room 124, all accessed from one side. FIG. 4C shows a layout with four washroom stalls, two female accessed from one side, and two male accessed from the other side, and the first aid room 124. FIG. 4D shows a layout with eight washroom stalls, four female on one side and four male on the other side, and the first aid room 124. Other layouts may be provided depending on the size of the trailers, the requirements of the staff, and the available access points at the trailer's location.

FIGS. 4E and 4F show alternative layouts with multiple stalls and access locations on both the end and side of the trailer system 100. More particularly, FIG. 4E shows a layout with two washroom stalls (one male, one female) access from the end of the trailer and a unisex washroom stall and first aid room accessible from one side. The first aid room 124 is enlarged to accommodate extra equipment as needed. FIG. 4F shows a similar layout, but with an extra access on the opposing side to an accessible space for storage, mechanical equipment, HVAC/electrical systems, etc.

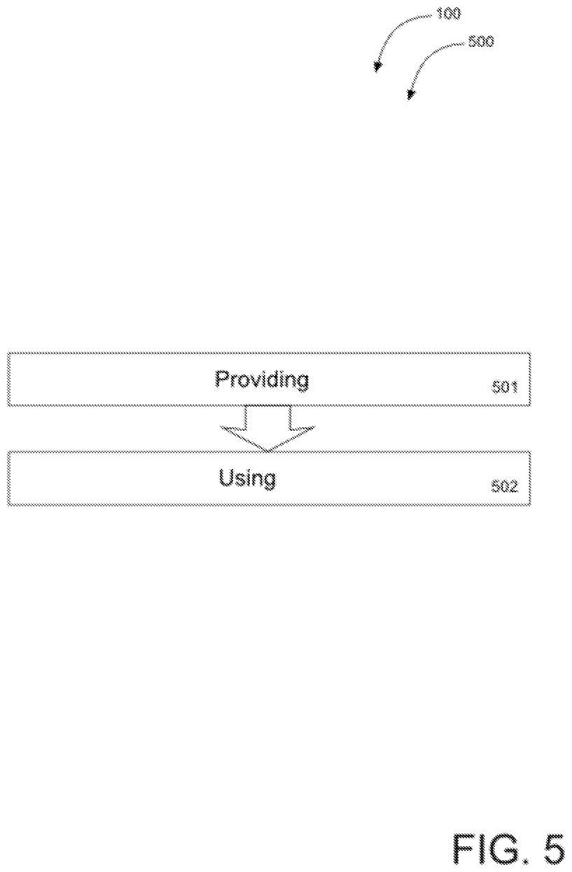

Referring now to FIG. 5 showing a flow diagram illustrating a method of use 500 for multi-functional movie industry trailer system 100, according to an embodiment of the present disclosure. In particular, the method of use 500 may include one or more components or features of the multi-functional movie industry trailer system 100 as described above. As illustrated, the method of use 500 may include the steps of: step one 501, providing a multi-functional movie industry trailer system comprising: a frame, an enclosure shell having at least one interior room, electrical wiring, and plumbing infrastructure; wherein the at least one interior room comprises at least two washrooms and a first-aid-room, and step two 502 using the multi-functional movie industry trailer system onsite for washroom-use and first-aid-use. In preferred embodiments the multi-functional movie industry trailer system is mobile.

It should be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for making trailers efficient are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A multi-functional movie industry trailer system comprising:
a frame;
an enclosure shell having
at least one interior room;
electrical wiring; and
plumbing infrastructure;
wherein said at least one interior room comprises at least two washrooms and a first-aid-room;
wherein said multi-functional movie industry trailer system comprises in functional combination said frame upon which said enclosure shell is mounted; and
wherein said enclosure shell comprises said at least one interior room, said at least two washrooms and said first-aid-room serviced by said electrical wiring and said plumbing infrastructure such that movie-related personnel can remain on-site.

2. The multi-functional movie industry trailer system of claim 1, wherein said at least one interior room comprises at least two washrooms, said first-aid-room and a living quarters.

3. The multi-functional movie industry trailer system of claim 1, wherein said first-aid-room comprises first-aid-supplies.

4. The multi-functional movie industry trailer system of claim 3, wherein said enclosure shell comprises exterior-walls.

5. The multi-functional movie industry trailer system of claim 4, wherein said enclosure shell comprises interior-walls.

6. The multi-functional movie industry trailer system of claim 1, wherein the frame is stationary.

7. The multi-functional movie industry trailer system of claim 1, wherein the frame comprises axles and wheels mounted thereto.

8. The multi-functional movie industry trailer system of claim 3, wherein the first-aid-supplies comprise at least plasters in a variety of different sizes and shapes; small, medium and large sterile gauze dressings; at least 2 sterile eye dressings; triangular bandages; crepe rolled bandages; safety pins; disposable sterile gloves; tweezers; scissors; alcohol-free cleansing wipes; sticky tape; thermometer; skin rash cream; cream and alternately spray to relieve insect bites and stings; antiseptic cream; painkillers; cough medicine; antihistamine tablets; distilled water; and a basic first aid manual.

9. The multi-functional movie industry trailer system of claim 4, wherein said first-aid-room comprises bathes and wash stations including a shower.

10. The multi-functional movie industry trailer system of claim 9, wherein the first-aid-room is accessible via a door in one said exterior-wall.

11. The multi-functional movie industry trailer system of claim 10, wherein the bathes and said wash stations are coupled to the plumbing infrastructure.

12. The multi-functional movie industry trailer system of claim 11, wherein the plumbing infrastructure transfers water.

13. The multi-functional movie industry trailer system of claim 1, wherein the first-aid-room comprises shelving.

14. The multi-functional movie industry trailer system of claim 1, wherein the electrical wiring is connected into the first-aid-room.

15. The multi-functional movie industry trailer system of claim 9, wherein the bathes and wash stations comprise valves.

16. The multi-functional movie industry trailer system of claim 1, wherein the frame comprises a hitch.

17. A multi-functional movie industry trailer system comprising:
a frame comprising axles and wheels mounted thereto;
an enclosure shell having
at least one interior room;
exterior-walls; and
interior-walls;
electrical wiring; and
plumbing infrastructure;
wherein said at least one interior room comprises at least two washrooms and a first-aid-room;
wherein said at least one interior room comprises at least said two washrooms, said first-aid-room and a living quarters;
wherein said first-aid-room comprises first-aid-supplies;
wherein said first-aid-room comprises bathes and wash stations including a shower;
wherein the first-aid-room is accessible via a door in one said exterior-wall;
wherein said multi-functional movie industry trailer system comprises in functional combination said frame upon which said enclosure shell is mounted; and
wherein said enclosure shell comprises said at least one interior room, said at least two washrooms and said first-aid-room serviced by said electrical wiring and said plumbing infrastructure such that movie-related personnel can remain on-site.

18. The multi-functional movie industry trailer system of claim 17, further comprising set of instructions; and
wherein the first-aid-supplies is arranged as a kit.

19. A method of use for the multi-functional movie industry trailer system, the method comprising the steps of:
providing a multi-functional movie industry trailer system comprising:
a frame;
an enclosure shell having
at least one interior room;
electrical wiring; and
plumbing infrastructure;
wherein said at least one interior room comprises at least two washrooms and a first-aid-room; and
using said multi-functional movie industry trailer system onsite for washroom-use and first-aid-use.

20. The method of claim 19, wherein said multi-functional movie industry trailer system is mobile.
